(19) United States
(12) Patent Application Publication
(10) Pub. No.: US 2023/0084628 A1
(43) Pub. Date: Mar. 16, 2023

(54) Use Of Radiation To Predispose Platelet Activation

(71) Applicant: CELL CODE, LLC, Mill Valley, CA (US)

(72) Inventors: Wayne J. Powell, Centennial, CO (US); Beth McDougall, Mill Valley, CA (US)

(21) Appl. No.: 17/802,108

(22) PCT Filed: Feb. 22, 2021
(86) PCT No.: PCT/US2021/018983

§ 371 Date: Aug. 24, 2022

Related U.S. Application Data
(60) Provisional application No. 62/980,550, filed on Feb. 24, 2020.

(51) Int. Cl. A61K 35/19; A61K 41/00; C12N 5/078

(52) U.S. Cl. CPC A61K 35/19; A61K 41/0023; C12N 5/0644; C12N 2529/00

(57) ABSTRACT

Methods for producing and utilizing primed platelets are provided, in which platelets are primed for release of specific granule types and/or active compounds by irradiation, for example with electromagnetic radiation, an electrical field, and/or a magnetic field. Such irradiation can be performed ex vivo or in vivo. Such primed platelets have utility in treating inflammatory conditions, neurodegenerative conditions, and/or joint and tendon related injuries.

This application claims the benefit of U.S. Provisional Patent Application No. 62/980,550 filed on Feb. 24, 2020. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is pre-activation of platelets.

### BACKGROUND

The following description includes information that may be useful in understanding the present invention. It is not an admission that any of the information provided herein is prior art or relevant to the presently claimed invention, or that any publication specifically or implicitly referenced is prior art.

Platelets are recognized as the primary cell regulating hemostasis and thrombosis, and are involved in inflammatory and immune responses during infection or injury. See Seong-Hoon Yun, et al, Platelet Activation: The Mechanisms and Potential Biomarkers, BioMed Research International, vol. 2016, Article ID 9060143, 5 pages, 2016. All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Platelets can be activated by thrombin through protease-activated receptors (PAR) on the platelet surface through G protein-coupled receptors (GPCR). Activated platelets secrete several inflammatory mediators, including various proteins, chemokines, cytokines, and growth factors. Platelet-Rich Plasma (PRP) has been used to favor angiogenesis processes, promote proliferation of undifferentiated stem cells, and accelerate bone formation. See Jaehoon Choi et al, Archives of Plastic Surgery 2012; 39(6):585-592.

Platelet granules are unique among secretory vesicles in both their content and their life cycle. Platelets contain three major granule types—dense granules, α-granules, and lysosomes (i.e., γ granules)—although other granule types have been reported (e.g., λ granules—contents involved in resorption during later stages of vessel repair.) Sharda A, Flaumenhaft R. The life cycle of platelet granules. F1000Res. 2018; 7:236. Published 2018 Feb. 28. doi:10.12688/f1000research.13283.1.

Previous work has used light irradiation on platelets. United States Patent Application Publication No. US 2017/0246470 A1 (Systems And Methods For Enhancing Platelet Biogenesis And Extending Platelet Lifespan With Low Level Light) by Meixiong Wu et al used low level light (LLL) to facilitate platelet biogenesis or extend platelet lifespan. United States Patent Application Publication No. US2010/0196497A1—Method of Treating Tissue Using Platelet-Rich Plasma in Combination with Low-Level Laser Therapy, by Lim et al, used platelet-rich plasma and laser energy (about 400 nm to 1500 nm, 1 mW to 500 mW, about 1 second to about 27.8 hours) to treat a patient's injured tissue.

Gres\check{ }ner et al. used green laser light irradiation on whole blood platelets and observed increased platelet cyclic GMP. The Effect Of Green Laser Light Irradiation On Whole Blood Platelets. Journal of Photochemistry and Photobiology B: Biology 79 (2005) 43-50. Prodouz K et al used visible light (450-600 nm) and a photosensitizer that resulted in a release of serotonin, aggregation, and marked morphological changes in platelets. Effects Of Two Viral Inactivation Methods On Platelets: Laser-UV Radiation And Merocyanine 540-Mediated Photoinactivation. Blood Cells. 1992; 18(1):101-14; discussion 114-6. Monika Olban et al used low power (1-5 J) red laser at 670 nm to trigger the release of substances stored in the specific granules. The Biostimulatory Effect Of Red Laser Irradiation On Pig Blood Platelet Function. Cell Biology International Volume 22, Issue 3, March 1998, Pages 245-248.

However, previous work does not teach using irradiation to prime platelets, i.e., pre-disposing the platelets by irradiation or other means, sufficiently to move target granules closer to the membrane surfaces, but not sufficiently to cause substantial release of the target granules.

Thus, there is still a need for new methods of irradiating platelets to prime the release of contents in specific granules.

### SUMMARY OF THE INVENTION

The inventive subject matter provides methods in which platelets are primed for release of specific granule types and/or active compounds by irradiation, for example with electromagnetic radiation, an electrical field, and/or a magnetic field. Such irradiation can be performed ex vivo or in vivo. Such primed platelets have utility in treating inflammatory conditions, neurodegenerative conditions, and/or joint and tendon related injuries.

One embodiment of the inventive concept is a method of treating a patient with a primed platelet by preparing a platelet rich solution that includes a platelet to be primed in suspension, subjecting the platelet to be primed to a field of irradiation energy in a manner sufficient to move target granules closer to a membrane surface of the platelet to be primed (but not sufficient to cause substantial release of the target granules) to generate the primed platelet, monitoring a concentration of molecule marker characteristic of a primed platelet in the platelet rich solution, and intravenously infusing the platelet rich solution into the patient before the molecular marker reaches a predetermined level. The irradiation energy is applied at a power between 0.1 mW and 1,000 mW, and is applied for between 0.1 second and 1 hour. In some embodiments the platelet to be primed is treated with a platelet activation inhibitor before and/or while being irradiated. Suitable platelet activation inhibitors include cyclooxygenase inhibitors, adenosine diphosphate (ADP) receptor inhibitors, phosphodiesterase inhibitors, protease-activated receptor-1 (PAR-1) antagonists, glycoprotein IIB/IIIA inhibitors, adenosine reuptake inhibitors, thromboxane inhibitors, and thromboxane synthase inhibitors.

Another embodiment of the inventive concept is a method of producing a primed platelet by obtaining a platelet rich solution comprising a platelet to be primed in suspension, and subjecting the platelet to be primed to a field of irradiation energy, in a manner sufficient to move target granules closer to a membrane surface of the platelet to be primed (but not sufficient to cause substantial release of the target granules). The irradiation energy is applied at a power between 0.1 mW and 1,000 mW, and is applied for between 0.1 second and 1 hour. In some embodiments the platelet to be primed is treated with a platelet activation inhibitor before and/or while being irradiated. Suitable platelet activation inhibitors include cyclooxygenase inhibitors, adenosine diphosphate (ADP) receptor inhibitors, phosphodiesterase inhibitors, protease-activated receptor-1 (PAR-1) antagonists, glycoprotein IIB/IIIA inhibitors, adenosine reuptake inhibitors, thromboxane inhibitors, and thromboxane synthase inhibitors.

Another embodiment of the inventive concept method of treating a patient with a primed platelet by irradiating a vascular portion of the patient with a field of irradiation energy, in a manner sufficient to move target granules closer to a membrane surface of a platelet to be primed (but not sufficient to cause substantial release of the target granules) to generate a primed platelet, monitoring (within in the patient or in a sample obtained from the patient) concentration of a molecule marker characteristic of a primed platelet, and halting irradiation the patient before the molecular marker exceeds a predetermined concentration. The irradiation energy is applied at a power between 0.1 mW and 1,000 mW, and is applied for between 0.1 second and 1 hour. In some embodiments the patient is treated with a platelet activation inhibitor before and/or while being irradiated. Suitable platelet activation inhibitors include cyclooxygenase inhibitors, adenosine diphosphate (ADP) receptor inhibitors, phosphodiesterase inhibitors, protease-activated receptor-1 (PAR-1) antagonists, glycoprotein IIB/IIIA inhibitors, adenosine reuptake inhibitors, thromboxane inhibitors, and thromboxane synthase inhibitors.

Various objects, features, aspects and advantages of the inventive subject matter will become more apparent from the following detailed description of preferred embodiments.

### DETAILED DESCRIPTION

The inventive subject matter provides apparatus, systems and methods in which low power irradiation energy (i.e., radiation or radiant energy) is used to prime platelets sufficiently to move target granules closer to the membrane surfaces, but not sufficiently to cause substantial release (i.e., greater than about 5%, 10%, or 25% of target granule content) of contents from the target granules. Different types of irradiation energy contemplated herein include electromagnetic energy (e.g., far infrared, infrared, visible light, ultraviolet, far ultraviolet, etc.), heat energy, electric fields, plasma radiation, acoustic waves (e.g., infrasound, audible sound, and/or ultrasound), and magnetic fields (e.g., as provided by permanent magnets, electromagnets, nuclear magnetic resonance (NMR) devices, etc.).

Irradiation energy can be applied continuously or discontinuously, and can be coherent or non-coherent. Similarly, irradiation energy can be polarized of have random polarization. Irradiation that is applied continuously can be applied at a constant intensity or, alternatively, intensity can be varied during a period of application. Such variation in intensity can be periodic, for example describing a wave form.

In some embodiments a single type of irradiation energy is applied. In other embodiments two or more types of irradiation energy are applied. In such embodiments the two or more types of irradiation energy can be applied in parallel or in a serial fashion.

Irradiation energy that is applied discontinuously can be applied in a periodic or non-periodic manner. Intensity of irradiation can be varied during a period of irradiation, and such variation can be periodic (e.g., following a waveform). Similarly, irradiation energy that is applied discontinuously can be applied such that the periods of irradiation describe a waveform. In some embodiments, variation in intensity in irradiation can be applied as a waveform that is superimposed on a second waveform that characterizes the periods of application.

Irradiation energy can be provided by any suitable device. For example, electromagnetic energy can be provided by one or more lasers, LEDs, or other sources of electromagnetic energy capable of emitting within the desired frequency range. Similarly, electric field can be provided by a suitable RF emitter, and magnetic fields can be provided by positioning of a magnetic source and/or application of power to an electromagnetic source. In some embodiments two or more sources can be provided in order to accommodate the application of a desired range or type of irradiation(s).

As used herein, the term “priming” as applied to platelets, means pre-disposing the platelets by irradiation or other energy, sufficiently to move target granules closer to the membrane surfaces, but not sufficiently to cause substantial release of contents from the target granules. Priming with respect to target granules does not preclude substantial release of contents from non-target granules.

One should appreciate that such priming provides a population of selectively responsive platelets, which in turn provide enhanced release of a desired active molecule upon stimulation. Such enhanced release increases the effectiveness of the platelet response, and provides new therapeutic modes.

Inventors believe that such primed platelets can be used to treat a variety of conditions, including inflammatory conditions (e.g., arthritis, tendinitis), neurodegenerative conditions, cardiovascular disease, and joint and/or tendon injuries.

Various objects, features, aspects and advantages of the inventive subject matter will become more apparent from the following detailed description of preferred embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

A platelet rich solution can be prepared according to Rachita Dhurat and MS Sukesh. Principles and Methods of Preparation of Platelet-Rich Plasma: A Review and Author's Perspective. J Cutan Aesthet Surg. 2014 October-December; 7(4): 189-197. Concentrated platelets can be prepared using the PRP (platelet-rich plasma) method:
1. Obtain whole blood by venipuncture in acid citrate dextrose (ACD) tubes
2. Do not chill the blood at any time before or during platelet separation.
3. Centrifuge the blood using a ‘soft’ spin.
4. Transfer the supernatant plasma containing platelets into another sterile tube (without anticoagulant).
5. Centrifuge tube at a higher speed (a hard spin) to obtain a platelet concentrate.
6. The lower ⅓rd is PRP and upper ⅔rd is platelet-poor plasma (PPP). At the bottom of the tube, platelet pellets are formed.
7. Remove PPP and suspend the platelet pellets in a minimum quantity of plasma (2-4 mL) by gently shaking the tube.

Alternatively, concentrated platelets can be prepared using the Buffy coat method:
1. Whole blood should be stored at 20° C. to 24° C. before centrifugation.
2. Centrifuge whole blood at a ‘high’ speed.
3. Three layers are formed because of its density: The bottom layer consisting of RBCs, the middle layer consisting of platelets and WBCs and the top PPP layer.
4. Remove supernatant plasma from the top of the container.
5. Transfer the buffy-coat layer to another sterile tube.
6. Centrifuge at low speed to separate WBCs or use leucocyte filtration filter.

In preferred embodiments, a platelet rich solution is subjected to a field of low power irradiation energy, sufficiently to move target granules closer to the membrane surfaces, but not sufficiently to cause substantial release of contents from the target granules. In some embodiments, the irradiation energy is applied at a power of from 0.1 mW to 1,000 mW (e.g., from 0.1 mW to 1 mW, 1 mW to 10 mW, 10 mW to 100 mW, 100 mW to 500 mW, and/or 500 mW to 1000 mW).

In some embodiments, the platelets are irradiated for a period of time ranging from about 0.1 second to about 1 hour. For example, platelets can be irradiation for from 0.1 to 1 second, 1 to 5 seconds, 5 to 10 seconds, 10 to 30 seconds, 30 to 60 seconds, 1 minute to 5 minutes, 5 minutes to 10 minutes, 10 minutes to 20 minutes, 20 minutes to 30 minutes, 30 minutes to 45 minutes, and/or 45 minutes to 60 minutes.

In some embodiments, the low power irradiation energy has a wavelength between about 1 nm and 10,000 nm. For example, the low power irradiation energy can have a wavelength of from 1 nm to 10 nm, 10 nm to 100 nm, 100 nm to 200 nm, 200 nm to 300 nm, 300 nm to 380 nm, 380 nm to 450 nm, 450 nm to 485 nm, 485 nm to 500 nm, 500 nm to 565 nm, 565 nm to 590 nm, 590 nm to 625 nm, 625 nm to 740 nm, 740 nm to 800 nm, 800 nm to 900 nm, 900 nm to 1,000 nm, 1,000 nm to 2,000 nm, 2,000 to 3,000 nm, 3,000 to 4,000 nm, 4,000 to 5,000 nm, 5,000 to 6,000 nm, 6,000 to 7,000 nm, 7,000 to 8,000 nm, 8,000 to 9,000 nm, and/or 9,000 nm to 10,000 nm.

It is contemplated that a combination of low power irradiation energy can be used to prime platelets, using any combination of wavelengths. In some embodiments, the irradiation energy comprises two or more frequency ranges. The irradiation energy field can also have an intensity gradient. A preferred irradiation energy is a laser beam. In especially preferred embodiments, the laser beam is phase conjugated. Moreover, the energy field can be pulsed.

In some embodiments, one or more platelet activation inhibitor(s) is(are) added to the platelet rich solution to prevent full activation of platelets. Such addition can occur prior to or during irradiation. Contemplated platelet inhibitors include antiplatelet drugs, for example, cyclooxygenase inhibitors (e.g., Aspirin, Triflusal (Disgren)), Adenosine diphosphate (ADP) receptor inhibitors (e.g., Cangrelor (Kengreal), Clopidogrel (Plavix), Prasugrel (Effient), Ticagrelor (Brilinta), Ticlopidine (Ticlid)), Phosphodiesterase inhibitors (e.g., Cilostazol (Pletaal)), Protease-activated receptor-1 (PAR-1) antagonists (e.g., Vorapaxar (Zontivity)), Glycoprotein IIB/IIIA inhibitors (e.g., Abciximab (ReoPro), Eptifibatide (Integrilin), Tirofiban (Aggrastat)), Adenosine reuptake inhibitors (e.g., Dipyridamole (Persantine)), Thromboxane inhibitors, Thromboxane synthase inhibitors, Thromboxane receptor antagonists (e.g., Terutroban).

In some embodiments, the concentration of a molecular marker for platelet priming in the platelet rich solution can be monitored in situ, for example using a chemical sensor. Alternatively, the platelet rich solution is sample periodically to measure the level of the molecular maker (for example, using a chemical or biochemical assay). Contemplated molecular markers for platelet priming include: P-selectin, platelet factor 4, transforming growth factor-β1, platelet-derived growth factor, fibronectin, B-thromboglobulin, Von Willebrand factor, fibrinogen, coagulation factor V, coagulation factor XIII, ADP, ATP, calcium, and serotonin.

Another embodiment of the inventive concept is a method for treating a patient using primed platelets. Such primed platelets are believed to be more responsive than unprimed platelets in regard to release of selected granules or granule types than unprimed or native platelets, and thus relatively more effective. In such methods platelets can be obtained from the individual to be treated (e.g., through venipuncture and treatment of the blood sample as described above), or can be obtained from an outside source. Suitable outside sources include another individual (e.g., through venipuncture or platelet donation) and products of cell culture.

If obtained from the patient, such platelets can be primed by ex vivo treatment of a platelet suspension obtained from the patient, for example via irradiation of a container enclosing the platelet suspension. The extent of priming can be determined by measurement of a marker characteristic of primed platelets in the platelet suspension. Such measurement can be continuous or periodic, as described above. The platelet suspension can undergo irradiation until the concentration of the marker reaches or approaches a predetermined cutoff value. The suspension, now containing primed platelets, can then be infused into the patient. Such infusion can be systemic (e.g., by introduction into a vein or artery) or localized (e.g., into or adjacent to an area to be treated).

Alternatively, in some embodiments primed platelets can be generated in vivo or within the patient. This can be accomplished by irradiating all or part of the patient at a frequency, intensity, and duration suitable to generate primed platelets. Suitable parameters for determining these values can be provided by obtaining one or more blood samples from the patient during treatment, isolating a platelet-rich fraction from the blood sample, and characterizing a marker associated with primed platelets in the platelet-rich fraction. Alternatively, as sensor can be instilled within the patient to provide such information. Irradiation can be continued for a defined interval, or until a concentration cutoff for the marker is approached or reached. In preferred embodiments, the irradiation is applied locally to a portion of the patient that is unpigmented and vascular (i.e., has a rich blood supply). Suitable locations include a mucous membrane, tympanic membrane, oral membrane, nasal membrane, and/or eye of the patient.

In some embodiments a patient can be treated with a platelet activation inhibitor prior to or during irradiation. Suitable platelet activation inhibitors are noted above.

It should be apparent to those skilled in the art that many more modifications besides those already described are possible without departing from the inventive concepts herein. The inventive subject matter, therefore, is not to be restricted except in the spirit of the appended claims. Moreover, in ‘interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1-81. (canceled)

82. A method of treating a patient with a primed platelet, comprising:
preparing a platelet rich solution comprising a platelet to be primed in suspension;
subjecting the platelet to be primed to a field of irradiation energy, in a manner sufficient to move target granules closer to a membrane surface of the platelet to be primed, but not sufficient to cause substantial release of the target granules, thereby generating the primed platelet; and
monitoring a concentration of molecule marker characteristic of the primed platelet in the platelet rich solution; and
intravenously infusing the platelet rich solution into the patient before the molecular marker reaches a predetermined level.

83. The method of claim 82, wherein the platelet to be primed is treated with a platelet activation inhibitor before being irradiated.

84. The method of claim 82, wherein the platelet to be primed is treated with a platelet activation inhibitor during irradiation.

85. The method of claim 83 or 84, wherein the platelet activation inhibitor is selected from the group consisting of a cyclooxygenase inhibitor and an adenosine diphosphate (ADP) receptor inhibitor.

86. The method of claim 83 or 84, wherein the platelet activation inhibitor is selected from the group consisting of a phosphodiesterase inhibitor and a protease-activated receptor-1 (PAR-1) antagonist.

87. The method of claim 83 or 84, wherein the platelet activation inhibitor is selected from the group consisting of a glycoprotein IIB/IIIA inhibitor and a glycoprotein IIB/IIIA inhibitor.

88. The method of claim 83 or 84, wherein the platelet activation inhibitor is selected from the group consisting of an adenosine reuptake inhibitor, a thromboxane inhibitor, and a thromboxane synthase inhibitor.

89. A method of producing a primed platelet, comprising:
obtaining a platelet rich solution comprising a platelet to be primed in suspension; and
subjecting the platelet to be primed to a field of irradiation energy, in a manner sufficient to move target granules closer to a membrane surface of the platelet to be primed, but not sufficient to cause substantial release of the target granules, thereby generating the primed platelet.

90. The method of claim 89, wherein the platelet to be primed is treated with a platelet activation inhibitor before being irradiated.

91. The method of claim 89, wherein the platelet to be primed is treated with a platelet activation inhibitor during irradiation.

92. The method of claim 90 or 91, wherein the platelet activation inhibitor is selected from the group consisting of a cyclooxygenase inhibitor and an adenosine diphosphate (ADP) receptor inhibitor.

93. The method of claim 90 or 91, wherein the platelet activation inhibitor is selected from the group consisting of a phosphodiesterase inhibitor and a protease-activated receptor-1 (PAR-1) antagonist.

94. The method of claim 90 or 91, wherein the platelet activation inhibitor is selected from the group consisting of a glycoprotein IIB/IIIA inhibitor, a thromboxane inhibitor, and a thromboxane synthase inhibitor.

95. A method of treating a patient with a primed platelet, comprising:
irradiating a vascular portion of the patient with a field of irradiation energy, in a manner sufficient to move target granules closer to a membrane surface of a platelet to be primed, but not sufficient to cause substantial release of the target granules, thereby generating the primed platelet; and
in the patient or in a sample obtained from the patient, monitoring a concentration of molecule marker characteristic of the primed platelet; and
halting irradiation of the patient before the molecular marker exceeds a predetermined level.

96. The method of claim 95, wherein the patient is treated with a platelet activation inhibitor before irradiation.

97. The method of claim 95, wherein the patient is treated with a platelet activation inhibitor during irradiation.

98. The method of claim 96 or 97, wherein the platelet activation inhibitor is selected from the group consisting of a cyclooxygenase inhibitor and an adenosine diphosphate (ADP) receptor inhibitor.

99. The method of claim 96 or 97, wherein the platelet activation inhibitor is selected from the group consisting of a phosphodiesterase inhibitor and a protease-activated receptor-1 (PAR-1) antagonist.

100. The method of claim 96 or 97, wherein the platelet activation inhibitor is selected from the group consisting of a glycoprotein IIB/IIIA inhibitor, a thromboxane inhibitor, and a thromboxane synthase inhibitor.
